(19) United States
(12) Patent Application Publication
(10) Pub. No.: US 2012/0289611 A1
(43) Pub. Date: Nov. 15, 2012

(54) PECTIN COMPOUNDS, METHODS OF USING PECTIN COMPOUNDS, AND METHODS OF CONTROLLING WATER SOLUBILITY

(76) Inventors: Daniel T. Daly, Tuscaloosa, AL (US); Scott K. Spear, Bankston, AL (US); Richard P. Swatloski, Tuscaloosa, AL (US)

(21) Appl. No.: 13/384,404

(22) PCT Filed: Sep. 29, 2010
(86) PCT No.: PCT/US10/50682

§ 371 Date: Jan. 17, 2012

Related U.S. Application Data
(60) Provisional application No. 61/247,080, filed on Sep. 30, 2009.

(51) Int. Cl. C08B 37/06; A01N 25/00; A61K 47/36

(52) U.S. Cl. 514/777; 536/2

(57) ABSTRACT

Briefly described, embodiments of the present disclosure provide for compositions including pectin compounds, pectin compounds, methods of making pectin compounds, methods of controlling the water solubility of a pectin compound, methods of controlling the water solubility of an agent, beads including pectin compounds, and the like.

### CROSS-REFERENCE TO RELATED APPLICATION

This application claims priority to U.S. provisional application entitled “PECTIN COMPOUNDS, METHODS OF USING PECTIN COMPOUNDS, AND METHODS OF CONTROLLING WATER SOLUBILITY,” having Ser. No. 61/247,080, filed on Sep. 30, 2009, which is entirely incorporated herein by reference.

### BACKGROUND

Pectin is a complex polysaccharide associated with plant cell walls, with the middle lamella layer of the cell wall the richest in pectin. Pectic substances are produced and deposited during cell wall growth and are particularly abundant in soft plant tissues under conditions of fast growth and high moisture content.

Pectin includes an alpha 1-4 linked polygalacturonic acid backbone intervened by rhamnose residues and modified with neutral sugar side chains and non-sugar components such as acetyl, methyl, and ferulic acid groups. The neutral sugar side chains, which include arabinan and arabinogalactans, are attached to the rhamnose residues in the backbone. The rhamnose residues tend to cluster together on the backbone.

The galacturonic acid residues in pectin are partly esterified and present as the methyl ester. The degree of esterification is defined as the percentage of carboxyl groups esterified. Pectin with a degree of esterification (“DE”) above 50% is named high methyl ester (“HM”) pectin or high ester pectin and one with a DE lower than 50% is referred to as low methyl ester (“LM”) pectin or low ester pectin.

### SUMMARY

Briefly described, embodiments of the present disclosure provide for compositions including pectin compounds, pectin compounds, methods of making pectin compounds, methods of controlling the water solubility of a pectin compound, methods of controlling the water solubility of an agent, beads including pectin compounds, and the like.

One exemplary composition, among others, includes a pectin compound having structure E

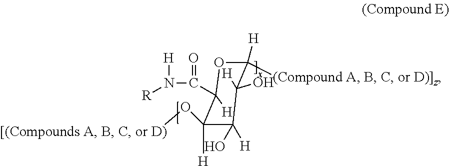

wherein R is a polyoxyalkyleneamine, wherein one or more of any one of compound structures A, B, C, or D can be included in compound E, wherein compound structures A, B, C, and D are the following:

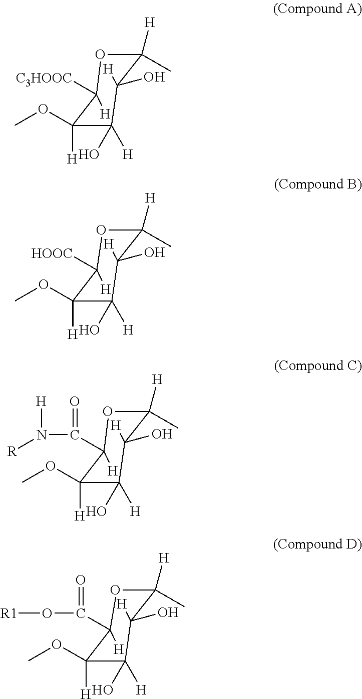

wherein each R1 is selected from an aliphatic group or a drug with an alcohol functionality, and wherein z is 300 to 800 and wherein x is 5 to 55.

One exemplary method of controlling the water solubility of a pectin compound, among others, includes: adjusting the ratio of the ester groups to the acid groups on the pectin compound, wherein the ratio of the ester groups to the acid groups determines the water solubility of the pectin compound, wherein if the lower the ratio the higher the water solubility of the pectin compound and the higher the ratio the lower the water solubility of the pectin compound.

One exemplary method of controlling the water solubility of a pectin compound, among others, includes: adjusting the ratio of the amide groups to the acid groups on the pectin compound, wherein the ratio of the amide groups to the acid groups determines the water solubility of the pectin compound, wherein if the lower the ratio the higher the water solubility of the pectin compound and the higher the ratio the lower the water solubility of the pectin compound.

One exemplary method of controlling the water solubility of a pectin compound, among others, includes: adjusting the ratio of one of: the amide groups to the acid groups, the ratio of the ester groups to the acid groups, or the ratio of the ester groups to the acid groups to the amide groups.

One exemplary method of controlling the water solubility of a pectin compound, among others, includes: adjusting the ratio of the ester groups to the acid groups on the pectin compound, wherein the ratio of the ester groups to the acid groups determines the water solubility of the pectin compound, wherein if the lower the ratio the lower the water solubility of the pectin compound and the higher the ratio the higher the water solubility of the pectin compound.

One exemplary method of controlling the water solubility of a pectin compound, among others, includes: adjusting the ratio of the amide groups to the acid groups on the pectin compound, wherein the ratio of the amide groups to the acid groups determines the water solubility of the pectin compound, wherein if the lower the ratio the lower the water solubility of the pectin compound and the higher the ratio the higher the water solubility of the pectin compound.

One exemplary method of controlling the water solubility of a pectin compound, among others, includes: altering the water solubility of the pectin compound to match the water solubility of the agent, wherein the water solubility is altered by adjusting the ratio of: the amide groups to the acid groups, the ratio of the ester groups to the acid groups, or the ratio of the ester groups to the acid groups to the amide groups.

### BRIEF DESCRIPTION OF THE DRAWINGS

Many aspects of this disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1.1 illustrates various chemical structures.

FIG. 1.2 illustrates various chemical structures.

FIG. 2.1 illustrates the effect of functionalization and degree of esterification of pectin.

FIG. 3.1 illustrates release rates of various pectin compounds.

FIG. 4.1 illustrates the spectroscopic data for the formation of amides.

FIG. 5.1 illustrates the release data for sodium salicylate a highly water soluble compound has a slower release rate when encapsulated with the more soluble LMP than the less soluble HMP. Likewise the lesser soluble salicylic acid has slower release rates in the less soluble pectins HMP, C12 esterfied pectin and LM104 versus the more soluble LMP, T-403 amide.

FIG. 6.1 illustrates some Jeffamine® amines.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, organic chemistry, pharmaceutical chemistry, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In this specification and in the claims that follow, reference will be made to a number of terms that shall be defined to have the following meanings unless a contrary intention is apparent.

As referred to herein and in the claims, “pectins” are a group of plant cell wall polymers—the rhamnogalacturonans. Rhamnogalacturonans are widely used in food and pharmaceutical industries for their versatile functional properties. They are anionic polysaccharides mainly of 1,4-linked α-D-galacturonic acid (GalA) residues and are classified either as high-methoxy (HM) or low-methoxy (LM) pectins. Pectins with a degree of esterification (DE) of GalA residues ≧50 are regarded as High-methoxy (HM) pectins, while those with DE <50 are low-methoxy (LM) pectins. Both types of pectins exhibit different rheological behavior as a result of their differing charge densities, resulting in numerous applications. Both HM and LM pectins are commonly used in tissue engineering, food production, and drug delivery systems.

Pectin can be derived from sources such as, but not limited to, fruit peels (e.g., citrus (e.g., oranges, limes, and the like), non-citrus (e.g., apples, tomato, pears, and the like), and the like), nuts (e.g., soy, peanut, sunflower, walnuts, and the like), vegetables (e.g., sugar beets, pumpkin, broccoli, onion, and the like), cacao, pine roots, and the like.

Pectin can have a molecular weight of about 60 to 130,000 g/mole.

The term “aliphatic group” refers to a saturated or unsaturated linear or branched hydrocarbon group and encompasses alkyl, alkenyl, and alkynyl groups, for example.

As used herein, “alkyl” or “alkyl group” refers to a saturated aliphatic hydrocarbon radical which may be straight or branched, having 1 to 20 carbon atoms, wherein the stated range of carbon atoms includes each intervening integer individually, as well as sub-ranges. Examples of alkyl groups include, but are not limited to, methyl, ethyl, i-propyl, n-propyl, n-butyl, t-butyl, pentyl, hexyl, septyl, octyl, nonyl, decyl, and the like.

As used herein, “alkenyl” or “alkenyl group” refers to an aliphatic hydrocarbon radical which may be straight or branched, containing at least one carbon-carbon double bond, having 2 to 20 carbon atoms, wherein the stated range of carbon atoms includes each intervening integer individually, as well as sub-ranges. Examples of alkenyl groups include, but are not limited to, ethenyl, propenyl, n-butenyl, i-butenyl, 3-methylbut-2-enyl, n-pentenyl, heptenyl, octenyl, decenyl, and the like.

The term “alkynyl” refers to straight or branched chain hydrocarbon groups having 2 to 12 carbon atoms, preferably 2 to 4 carbon atoms, and at least one triple carbon to carbon bond, such as ethynyl. An alkynyl group is optionally substituted, unless stated otherwise, with one or more groups, selected from aryl (including substituted aryl), heteroaryl, heterocyclo (including substituted heterocyclo), carbocyclo (including substituted carbocyclo), halo, hydroxy, alkoxy (optionally substituted), aryloxy (optionally substituted), alkylester (optionally substituted), arylester (optionally substituted), alkanoyl (optionally substituted), aroyl (optionally substituted), cyano, nitro, amino, substituted amino, amido, lactam, urea, urethane, sulfonyl, and the like.

The terms “halo” and “halogen” refer to the fluoro, chloro, bromo or iodo groups. There can be one or more halogen groups, which can be the same or different. In an embodiment, each halogen can be substituted by one of the other halogens or a hydrogen group. The term “substituted” includes substituting a halogen for a hydrogen atom in one or more places.

### GENERAL DISCUSSION

Embodiments of the present disclosure provide for compositions including pectin compounds, pectin compounds, methods of making pectin compounds, methods of controlling the water solubility of a pectin compound, methods of controlling the water solubility of an agent, beads including pectin compounds, and the like.

Embodiments of the present disclosure are advantageous because the water solubility of the pectin compound can be adjusted, which allows for the production of pectin beads and encapsulation of agents such as pharmaceuticals, pesticides, or a nutriceuticals, and the like. In an embodiment, the water solubility of the pectin compound and the agent can be matched so that the release rate of the agent can be precisely controlled.

Embodiments of the present disclosure allow the composition including the pectin compound or the pectin compound to be customized for different applications (e.g., slow delivery, moderate delivery, fast delivery) and/or agents (e.g., active ingredients (e.g., drugs)). Embodiments of the present disclosure may find application in the encapsulation market, agent delivery market, and the like, for the drug market and the agricultural market (e.g., release of pesticides, herbicides, fertilizers, seeds, and the like).

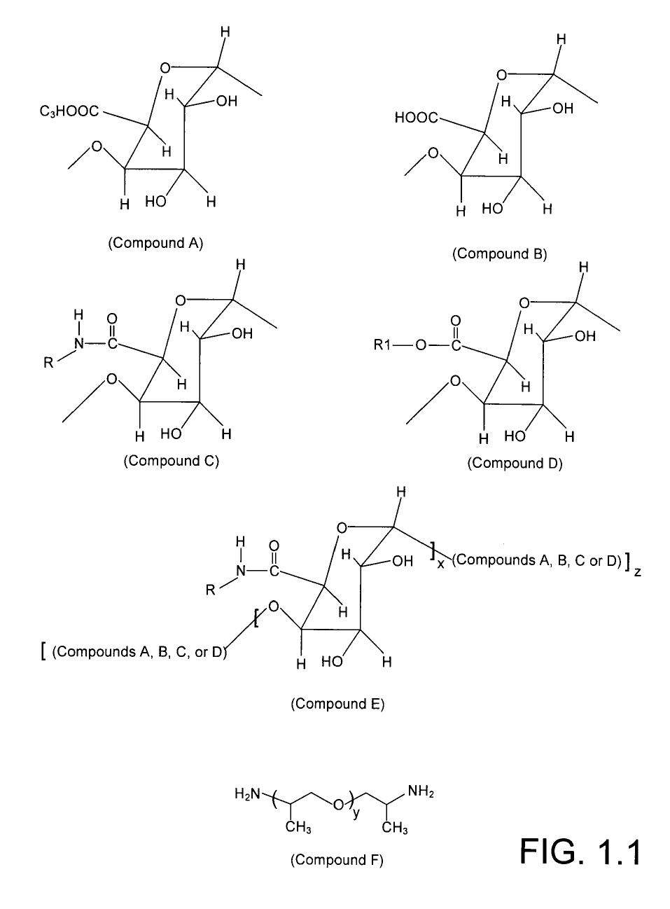

Embodiments of the present disclosure include pectin compounds and compositions or beads that include a pectin compound. In an embodiment, the pectin compound can include a compound having structure E in FIG. 1.1. R can be a polyoxyalkyleneamine and x can be 5 to 55 or about 7 to 30.

As shown in FIG. 1.1, the pectin compound can include a structure having the core structure of compound C along with addition of zero or one or more of compound structures A, B, C, D, or combinations thereof, shown in FIG. 1.1, in any order on one or both sides of the core. Any one of compound structures A, B, C, or D and any combination of them (e.g., random, repeating units, etc) can be attached to one or both sides of the core compound C. The units or combinations of compound structures on each side of the core can be the same or different. R1 can be an aliphatic group (e.g., C1 to C20) or a drug having alcohol functionality. In another embodiment, the pectin compound can have a core structure of compound D along with compound structures A, B, C, or D. In an embodiment, compound E or H can have multiple repeating units (e.g., z=300 to 700) including the core structure of compound C (alternatively core structure of compound D).

As mentioned above, embodiments of the present disclosure provide for the ability to control the water solubility of the pectin compound. In an embodiment, the water solubility of the pectin compound can be controlled by modifying the ester group of the pectin molecule. In this regard, the ratio of the ester groups, acid groups, and/or amide groups can be adjusted to produce a pectin compound having the desired water solubility properties (e.g., matching the water solubility of the pectin compound to an agent encapsulated in the pectin compound bead).

Embodiments of the present disclosure provide for the ability to control the ratio of the ester groups to the acid groups in a pectin compound. The ratio can be about 10:90 to 85:15 or about 25:75 to 75:25.

Embodiments of the present disclosure provide for the ability to control the ratio of the amide groups to the acid groups in a pectin compound. The ratio can be about 15:85 to 75:25 or about 20:80 to 60:40.

Embodiments of the present disclosure provide for the ability to control the ratio of the ester groups to the amide groups in a pectin compound. The ratio can be about 60:20 to 10:50 or about 40:20 to 20:40.

In an embodiment, the pectin group can have a ratio of the ester groups to the acid of about 10:90 to 85:15 or about 25:75 to 75:25, a ratio of the amide groups to the acid groups of about 15:85 to 75:25 or about 20:80 to 60:40, and/or a ratio of the ester groups to the amide groups of about 60:20 to 10:50 or about 40:20 to 20:40.

In an embodiment, the pectin compound can be modified by a reaction of the ester and or carboxylic acid group with a polyoxyalkyleneamine. The ester is formed by the acid catalyzed reaction between and alcohol and the carboxylic acid group.

In addition to modifying the ratio of the various groups, other compounds can be added to the pectin compound to form a bead, for example, to adjust the water solubility or the release rate of the agent. In an embodiment, calcium acetate can be added to the composition including the pectin compound. In addition, compounds such as zinc acetate, magnesium acetate, ZnCl₂, or MgCl₂, can be used to adjust the water solubility or the release rate of the agent.

In an embodiment, a polyoxyalkyleneamine can contain primary amino groups attached to the terminus of a polyether backbone. The polyether backbone is based either on propylene oxide, ethylene oxide, butlylene oxide, or a combination thereof. The polyoxyalkyleneamines can be monoamines, diamines, and triamines, having a molecular weight up to about 5000 amu. FIG. 1.1 illustrates an embodiment of a polyoxyalkyleneamine (compound F), where y can be 5 to 55 or about 7 to 30. In an embodiment, the polyoxyalkyleneamine (compound F) has a molecular weight of about 250 to 4000 amu. A type of polyoxyalkyleneamine is referred to as a Jeffamine®. Jeffamine® diamines (e.g., D series, MW from about 200 to 5000 amu), triamines (e.g., T series, MW from about 400 to 6000 amu), and secondary amines (e.g., SD series, MW from about 300 to 3000 amu) can be used. Additional details can be obtained from Huntsman Corporation. A number of illustrative types of polyoxyalkyleneamines are listed in the table below.

| SURFONAMINE ® | | Ratio PO/EO | Approx. |
|---|---|---|---|
| surfactant amine | Structure | y/x | Mol. Wt. |
| B-60 | CH₃—[OCH₂—CH₂]ₓ—[OCH₂CH(CH₃)]_{y}—NH₂ | 9/1 | 600 |
| L-100 | CH₃—[OCH₂—CH₂]ₓ—[OCH₂CH(CH₃)]_{y}—NH₂ | 3/19 | 1,000 |
| B-200 | CH₃—[OCH₂—CH₂]ₓ—[OCH₂CH(CH₃)]_{y}—NH₂ | 29/6 | 2,000 |
| L-207 | CH₃—[OCH₂—CH₂]ₓ—[OCH₂CH(CH₃)]_{y}—NH₂ | 10/31 | 2,000 |
| L-300 | CH₃—[OCH₂—CH₂]ₓ—[OCH₂CH(CH₃)]_{y}—NH₂ | 8/58 | 3,000 |
| B-30 | CH₃(CH₂)₁₂—OCH₂CH(CH₃)—OCH₂CH(CH₃)—NH₂ | — | 325 |
| Chemical Intermediate | | | |
| B-100 | 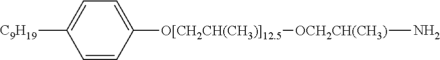 | — | 1004 |

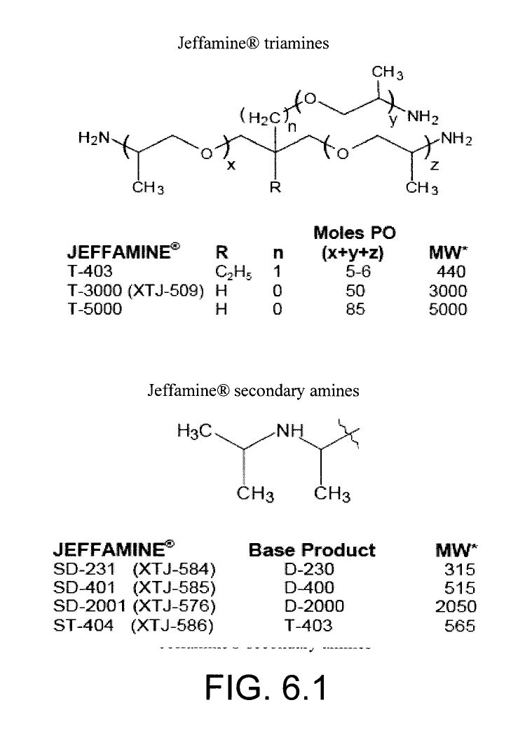

FIG. 6.1 illustrates Jeffamine® triamines and Jeffamine® secondary amines. Jeffamine® triamines series of compounds are triamines prepared by reaction of PO with a triol initiator followed by amination of the terminal hydroxyl groups. Jeffamine® secondary amines series are prepared by reacting a ketone with the amine end-groups of a secondary diamine (SD) or a secondary triamine (ST). Then it is reduced to create hindered secondary amine end groups represented by the structure shown in FIG. 6.1 (bottom structure). One reactive hydrogen on each end group provides for more selective reactivity and makes these secondary di- and triamines useful for intermediate synthesis and intrinsically slower reactivity compared with the primary Jeffamine® amines. Product information regarding Jeffamine® amines are described in FIG. 6.1.

In an embodiment, the pectin compound can include compound H as shown in FIG. 1.2. Compound H includes the polyoxyalkyleneamine (compound F) shown in FIG. 1.2, where y can be 5 to 55 or about 7 to 30.

Embodiments of the present disclosure can include pectin compounds including an agent (e.g., a drug (e.g., a small molecule drug) a pesticide, or a nutriceutical). In an embodiment, the agent is encapsulated by the pectin compound during the formation of beads made of the pectin compound. In an embodiment the bead can be made using spray drying, for example. In an embodiment the pectin compound can be designed to release at a certain rate (e.g., fast, medium, slow). In an embodiment, the water solubility of the pectin compound and the agent can be matched so that the release of the agent can be carefully and deliberately controlled. Additional details are described in Example 1. The table below shows some solubilities of pectins. Drugs typically have a lower solubility.

Solubility of Soy Hull Pectin Extracted at Different
Hull/Solvent Ratios, of Commercial Food-Grade Pectins

| Samples | 2.0 | 4.0 | 6.0 | 8.0 | 10.0 |
|---|---|---|---|---|---|
| Soy pectin (1:10) | 1.98 ± 0.07 | 1.93 ± 0.24 | 1.90 ± 0.18 | 1.44 ± 0.05 | 1.77 ± 0.14 |
| Soy pectin (1:15) | 1.80 ± 0.35 | 1.61 ± 0.07 | 1.73 ± 0.14 | 1.56 ± 0.14 | 1.62 ± 0.13 |
| Soy pectin (1:20) | 1.91 ± 0.09 | 1.98 ± 0.12 | 1.73 ± 0.03 | 1.89 ± 0.07 | 1.73 ± 0.08 |
| Soy pectin (1:25) | 1.83 ± 0.16 | 1.73 ± 0.21 | 1.73 ± 0.15 | 1.64 ± 0.13 | 1.63 ± 0.12 |
| Commercial pectin I HMP | 1.27 ± 0.06 | 1.29 ± 0.20 | 1.50 ± 0.14 | 1.35 ± 0.03 | 1.36 ± 0.14 |
| Commercial pectin IILMP | 2.24 ± 0.16 | 2.49 ± 0.07 | 2.54 ± 0.07 | 2.52 ± 0.12 | 2.55 ± 0.17 |
| Citrus pectin (Sigma) LMP | 1.50 ± 0.04 | 1.72 ± 0.23 | 1.78 ± 0.14 | 1.62 ± 0.07 | 1.75 ± 0.19 |

I (DE = 76.2) and II (DE = 32), and Analytical-Grade Pectin (citrus pectin; Sigma Chemical Co., St. Louis, MO)
Solubility (%) of pectins at different pH
aValues with same roman letter in each row and same roman superscript in each column are not significantly different (P < 0.05) from each other.

It should be noted that the solubility of pectin HMP is 1.27 g/100 ml or 0.013 g/ml, whereas LMP is 2.24 g/100 ml or 0.0224 g/ml.

The following are some solubilities of a number of drugs: Sodium alendronate (solubility of 1 mg/L in water), Celecoxib (Very low water solubility (3.3 mg/L)), Atorvastatin Calcium (sodium salt soluble in water, 20.4 ug/mL (pH 2.1), 1.23 mg/mL (pH 6.0)), Losartan (solubility of 0.82 mg/L in water), Fexofenadine Hydrochloride (freely soluble in methanol and ethanol, slightly soluble in chloroform and water, and insoluble in hexane), Carvedilol (practically insoluble (0.583 mg/L)), Mometasone furoate (practically insoluble), potassium losartan (0.82 mg/L), Atorvastatin Cacium (Sodium salt soluble in water, 20.4 ug/mL (pH 2.1), 1.23 mg/mL (pH 6.0)), Levofloxacin (Insoluble), Telmisartan (practically insoluble), Anastrozole (0.5 mg/mL), Zoledronic acid monohydrate (sparingly soluble), Olanzapine (practically insoluble in water), Esomeprazole (very slightly soluble in water), Lansoprazole (solubility of 0.97 mg/L in water), Risperidone (solubility of 2.8 mg/L in water), Clopidogrel bisulphate (solubility of 50.78 mg/L in water), Valsartan (soluble in ethanol and methanol and slightly soluble in water), Clopidogrel bisulphate (solubility of 50.78 mg/L in water), Citalopram Hydrobromide (31 mg/L), Cetirizine Hydrochloride (101 mg/L), Pioglitazone hydrochloride (slightly soluble in anhydrous ethanol, very slightly soluble in acetone and acetonitrile, practically insoluble in water, and insoluble in ether), Conjugated estrogenic hormones (0.0036 mg/ml), Ramipril (3.5 mg/L), and Fluticasone propionate (0.51 mg/L (insoluble)). Based on this information, the solubility of the pectin compound and the drugs can be aligned with one another so that they are similarly soluble.

An embodiment of the present disclosure provides for methods of controlling the water solubility of a pectin compound. The method can include adjusting the ratio of the ester groups to the acid groups on the pectin compound. The ratio of the ester groups to the acid groups determines, at least in part, the water solubility of the pectin compound. The lower the ratio of the ester groups to the acid groups, the higher the water solubility of the pectin compound. The higher the ratio of the ester groups to the acid groups, the lower the water solubility of the pectin compound. Thus, by adjusting the ratio, the solubility of the pectin compound can be controlled. As mentioned above, the ratio can be adjusted by a reaction of the polyoxyalkyleneamine with the ester group of the pectin compound.

In an embodiment, adjusting includes reaction of the pectin compound with a polyoxyalkyleneamine compound so that the polyoxyalkyleneamine compound displaces the ester group of the pectin compound. In another embodiment, adjusting includes reaction of the pectin compound with an aliphatic alcohol so that the carboxylic acid is converted into an ester group of the pectin compound.

An embodiment of the present disclosure provides for methods of controlling the water solubility of a pectin compound. The method includes adjusting the ratio of the amide groups to the acid groups on the pectin compound. The ratio of the amide groups to the acid groups determines, at least in part, the water solubility of the pectin compound. If the ratio of the amide groups to the acid groups (e.g., 15:85) is lower, then the water solubility (e.g., 0.01 g/ml) of the pectin compound is higher. If the ratio of the amide groups to the acid groups (e.g., 60:40) is higher, then the water solubility (e.g., 0.005 g/ml) of the pectin compound is lower.

In an embodiment, adjusting includes reaction of the pectin compound with a polyoxyalkyleneamine compound so that the polyoxyalkyleneamine compound displaces the ester group of the pectin compound. In another embodiment, adjusting includes reaction of the pectin compound with a polyoxyalkyleneamine compound so that the polyoxyalkyleneamine compound cleaves the ester group of the pectin compound forming an amide In another embodiment, adjusting includes reaction of the pectin compound with a polyoxyalkyleneamine compound so that the polyoxyalkyleneamine compound is converted into the amide group of the pectin compound.

An embodiment of the present disclosure provides for methods of controlling the water solubility of an agent in a pectin compound. The method includes altering the water solubility of the pectin compound to match the water solubility of the agent. The water solubility is altered by adjusting the ratio of either the amide groups to the acid groups, the ratio of the ester groups to the acid groups, or the ratio of the ester groups to the acid groups to the amide groups.

Additional embodiments of the present disclosure are described in the Examples.

### EXAMPLES

Now having described the embodiments of the present disclosure, in general, the Examples describe some additional embodiments of the present disclosure. While embodiments of present disclosure are described in connection with the Examples and the corresponding text and figures, there is no intent to limit embodiments of the present disclosure to these descriptions. On the contrary, the intent is to cover all alternatives, modifications, and equivalents included within the spirit and scope of embodiments of the present disclosure.

### Example 1

These Examples describe the rate of platinum-(II), Pt-(II), release from high-methoxy and low-methoxy pectin beads as well as N-polyoxyetheramine pectinamides. The release kinetics of Pt-(II) was studied in deionized water at room temperature and measured using atomic absorption (AA) spectroscopy. Pectin-Pt-(II) beads were prepared by a spray-drying method. It has been speculated that Pt-(II) release from pectin beads could be selectively controlled using pectin with differing degrees of esterification (DEs). Knowledge of the kinetics of Pt-(II) dissolution provides invaluable clues to elucidate controlled-release materials for biological applications. This Example describes the results of dissolution studies for a series of Pectin-Pt-(II) beads.

Jeffamines® are a family of polyether compound products (see compound E in FIG. 1.1). They are composed of a polyether backbone with a primary amino group attached at the terminus end as is the case for D and T series, but can also be secondary amines, SD series. There are multiple series that comprise the Jeffamine® family. Known for their flexibility, Jeffamines® are readily able to undergo reactions with esters to create amide compounds. Amidated pectin derivatives were prepared from highly methoxylated citrus pectin by the treatment with Jeffamine® D-230 (230 MW).

### Experimental

### Materials

LM pectin with DE of 9% and cis-diamminedichloroplatinum(II), also known as cisplatin, were purchased from Aldrich (Milwaukee, Wis.). The HM pectin with DE of 72% (GENU Pectin type B) and LM pectin of 28% (GENU Pectin type LM-104 AS-FS) were obtained from CP Kelco (Lille Skensved, Denmark). LM pectin, HM pectin, and LM pectin of DE 28% are referred to as LMP, HMP, and LMP-104, respectively.

### Procedure for Synthesis of Pectin-Jeffamine D-230 Conjugate

Two grams of HM pectin (DE ˜72%) were measured out and placed into a 150 ml round bottom flask with a small stir bar. 50 ml of distilled dimethylformamide was accurately measured in a graduated cylinder and added to the flask. A stoichiometric amount of Jeffamine® D-230 was weighed in a small beaker, and 50 mL freshly distilled dimethylformamide was mixed with the Jeffamine® and all was poured into the round bottom flask. The flask was heated to 60° C. and placed on a constant stir. The reaction was allowed to run for 24 hours followed by filtering the sample and rinsing with methanol then air dried. Bead samples prepared are referred to as HMP+Amide.

### Procedure for Production of Pectin-Pt-(II) Bead

Three grams of pectin was dissolved in 125 mL deionized water at 85° C. with magnetic stirring. Solutions were cooled to room temperature prior to adding 18 mL cisplatin solution (1 mg/mL, total 0.6 wt % on pectin) with continued stirring. Calcium acetate (2 wt % on LM pectin) was added at this point for sample referred to as LMP+ca. Beads were formed using a Büchi Mini Spray Dryer Model B-290 with inlet temperature at 150° C., aspirator at 100%, peristaltic pump at 15%, and pressurized air at 40 mm. The powders were stored dry at room temperature in closed containers.

### Procedure for Pectin-Pt-(II) Dissolution

Platinum release from pectin-Pt-(II) powder was studied by using a dissolution tester (Caframo BDC 1850) at a stirring speed of 150 rpm. Weighed amounts of pectin-Pt-(II) beads were tested using 30 mL of dissolution medium (deionized water maintained at room temperature). An aliquot (1 mL) of the release medium was collected using a Finnpipette (H79195) at predetermined time intervals and an equal amount of deionized water at room temperature was replaced. Pt-(II) release was expressed by percentage of Pt-(II) loss relative to the total mass of encapsulated Pt-(II).

### Determination of Pt-(II) Concentration

All samples prepared from pectin-Pt-(II) dissolution were analyzed using atomic absorption (AA) spectroscopy (Perkin Elmer). 1 mL of 21% sulfuric acid and 1 mL of water were added to each aliquot to completely hydrolyze all glycosidic linkages in the pectin polymer. A calibration curve was prepared from cis-diamminedichloroplatinum(II), and the concentration of platinum in each sample was determined by interpolation from the calibration curve.

### Discussion

The actual versus theoretical loading of Pt-(II) in each of the bead samples is shown in Table 1. In general, the spray drying method results in beads containing the desired amount of cisplatin.

TABLE 1
Example 1. Pt-(II) Loading of Pectin Beads

| Formulation | Theoretical wt % | Actual wt % |
|---|---|---|
| HMP | 0.60 | 0.56 |
| LMP | 0.60 | 0.63 |
| LMP + ca | 0.60 | 0.74 |
| HMP + Amide | 0.60 | 0.67 |
| LMP-104 | 0.60 | 0.61 |

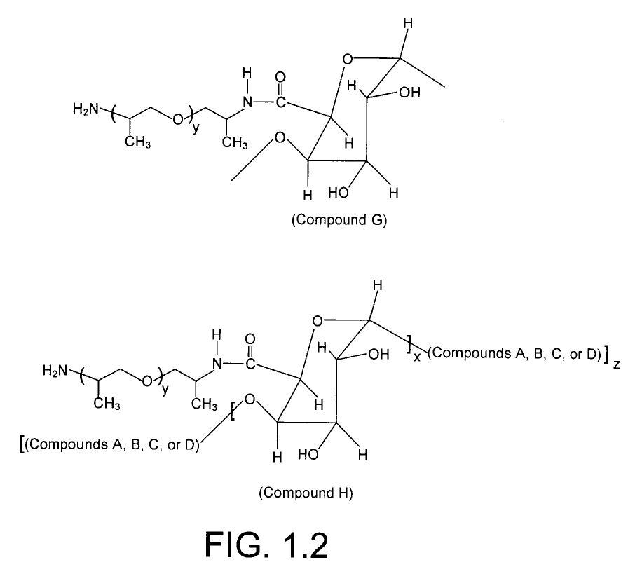

The rate of platinum release from pectin was observed to increase initially and slow as time elapsed. A typical plot from this dissolution experiment is shown in FIG. 2.1. As shown on the graph, the rate of release and percent theoretical yield of Pt-(II) are affected by the degree of esterification as well as presence of calcium ions from calcium acetate and polyoxyetheramine functionalization. The above experiments were only shown for five hours, but other data shows that the slope of the release rate stay constant until all of the cisplatin is released.

FIG. 2.1 shows the concentration of cisplatin versus time of a range of solubility of pectins and there encapsulation efficiency. The most soluble pectin has the lowest release rate or the lowest concentration of cis-platin since LMP-104 is vey soluble in water. Whereas the most non-soluble pectin gave the highest release rates since it has the lowest encapsulation efficiency. The extrapolation is that during the bead making process. The insoluble pectins will start forming beads prior to encapsulation and while the drug is still soluble in the evaporating water, forcing the drug to bind to the surface of the bead. The most soluble pectins will not start encapsulation until the drug begins to become insoluble allowing more drug to be captured into the bead.

### Example 2

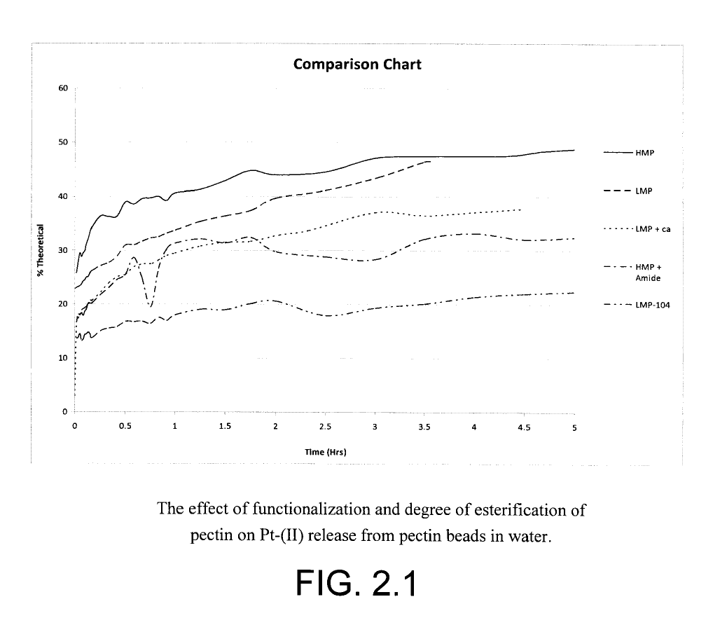

In this Example we examine the release rates of various pectin compounds (See FIG. 3.1). This example shows a release study showing the release of a water soluble amine. The faster release with High Methoxylated Pectin (HMP) versus Low Methoxylated Pectin (LMP) is consistent with embodiments of the present disclosure since the matching of water solubility's of the pectin and the compound will enhance encapsulation. It should be noted that better encapsulation results in slower release. This is due to the need of the compound being encapsulated to adhere to the polysaccharide during bead formation. If the polysaccharide becomes insoluble first during bead formation then the compound will adhere to the exterior of the bead resulting in faster release.

From FIG. 3.1 it can be seen that the water soluble Jeffamine has a higher concentration and release rate (small square, lower curve) when place in the HMP bead versus the LMP (large square, upper curve). Also notice the amount of beads in solution is higher (diamond) for the LMP than for the HMP (triangle) since LMP beads are more water soluble.

### Example 3

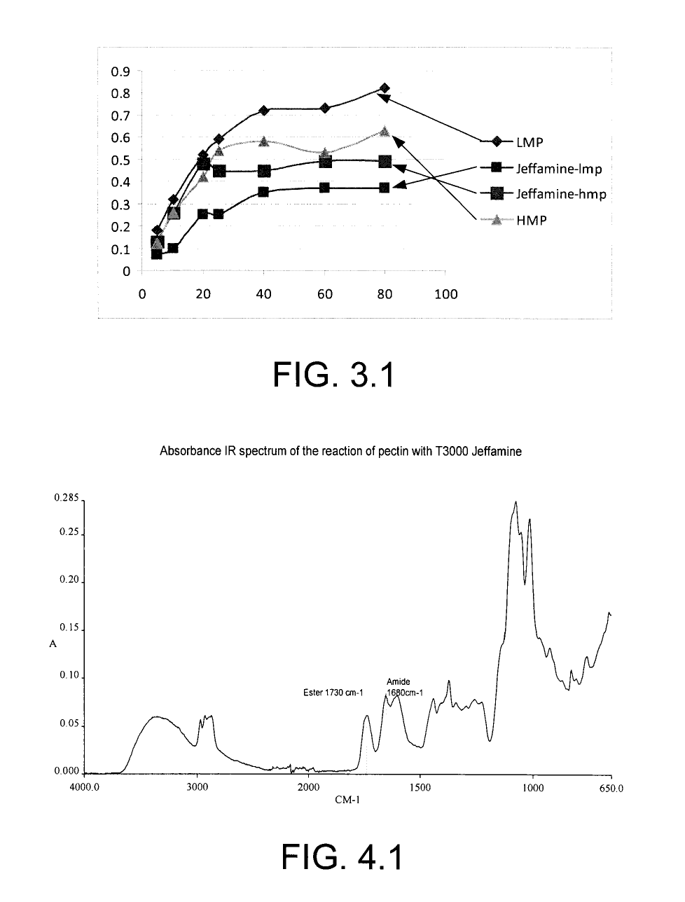

In this Example we discuss how the amounts of each of ester, amide and acid can be determined. FIG. 4.1 illustrates the spectroscopic data for the formation of amides. We have reacted HMP with Jeffamines®. We used the ratio of absorbencies of the ester (1730 cm⁻¹) to the amide (1680 cm⁻¹) of commercial amide GENU-L104 which reports a ratio of 26% ester to 22% amide. Based on this ratio the percent ester to amide would be 26.6% for Jeffamine® T-300 and 27.4% for Jeffamine® 230 and 41.74% for Jeffamine® 2001.

### Example 4

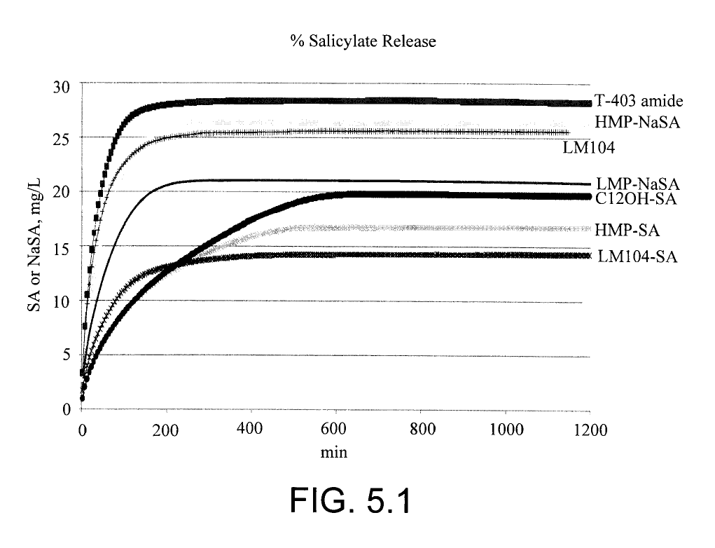

In this example we discuss the matching of the solubility's of the modified pectin to the drug salicylic acid (SA) and the salt sodium salicylate (NaSA). FIG. 5.1 illustrates the release data for sodium salicylate a highly water soluble compound has a slower release rate when encapsulated with the more soluble LMP than the less soluble HMP. Likewise the lesser soluble salicylic acid has slower release rates in the less soluble pectins HMP, C12 esterfied pectin and LM104 versus the more soluble LMP, T-403 amide. Thus, in this example LMP 104-SA is the commercial pectin with encapsulated salicylic acid and has a DE of 26%, HMP-SA is a commercial pectin from CP Kelco having a DE of 71.5%, LMP-NaSA is a commercial pectin from Aldrich having a DE of 8.9% and encapsulated with sodium salicylic acid, LM104 is a commercial pectin from CP Kelco having a DE of 26%, HMP-SA is a commercial pectin from CP Kelco having a DE of 71.5%) having an encapsulated sodium salicylic acid, C12OH-SA is HMP (a commercial pectin from CP Kelco having a DE of 71.5%) which has been transesterified with a c12 alcohol group and with encapsulated salicylic acid, and T-403 is HMP (a commercial pectin from CP Kelco having a DE of 71.5%) which has been reacted with Jeffamine T-403.

### Drug Release Studies

The release of salicyclic acid or sodium salicylate from pectin microspheres was investigated in deionized water. Dissolution studies were carried out using USP basket apparatus (type I) at a rotational speed of 150 rpm at 25° C. Microspheres weighing approximately 200 mg were gently folded into a small piece of Kimwipe™ and placed inside the USP basket apparatus. The release kinetics were monitored at 296 nm using a Cary-3C UV-Vis spectrophotometer over a period of 24 hours. Concentration of salicylic acid or sodium salicylate released was determined by method of standard addition.

### Example 5

### Pectin Solubility

### Conditions:

In de-ionized water at room temperature (about 22° C. of solution) with rotary mixing.

### Method:

0.11 g of solute was added to 8.0 mL de-ionized water in a test tube with screw cap and rotary mixed for 24 hours. The test tube with solute/solvent was then centrifuged for 2 minutes and 5.0 mL of sample were transferred to a glass vial and oven dried to constant mass at 70° C.

### Samples:

Pectin from citrus (Aldrich) DE=8.9%; LMP-C

Pectin from apple (Aldrich) DE=9.9%; LMP-A

Genu pectin 150 (CP Kelco) DE=71.5%; HMP

Jeffamine D230 Pectinamide DE=15%; D230-HMP

Jeffamine T403 Pectinamide DE=14%; T403-HMP

Jeffamine T3000 Pectinamide DE=30%; T3000-HMP

Jeffamine SD2001 Pectinamide DE=33%; SD2001-HMP

TABLE 1
Example 5

| | Solubility | Total time to |
|---|---|---|
| | (solute/solvent), | reach the |
| Sample | g/mL | saturation, h |
| LMP-C* | 0.0136 | 24 hrs |
| LMP-A* | 0.0166 | 24 hrs |
| HMP | 0.0125 | 24 hrs |
| D230-HMP | 0.0112 | 24 hrs |
| T403-HMP | 0.0109 | 24 hrs |
| T3000-HMP | 0.009 | 24 hrs |
| SD2001-HMP* | 0.0175 | 24 hrs |

*0.21 g solute used for solubility test.

In DI water, room temperature (22° C. of solution), magnetic stirring

### Samples:

Pectin from citrus (Aldrich) DE=8.9% (LMP)

Pectin from apple (Aldrich) DE=9.9% (LMP)

Genu pectin 150 (CP Kelco) DE=71.5% (HMP)

Genu pectin LM-104 AS-FS(CP Kelco) DE˜26% & degree of amidation˜22% (Amidated LMP)

Pectin from Aldrich, DE=8.9%, PEG having a molecular weight of 200 AMU, the PEG attached to the pectin by an ester, Poly (ethylene glycol) functionalized LMP (LMP/PEG-200)

Pectin from Aldrich, DE=8.9%, PEGME having a molecular weight of 700 AMU, the PEGME attached to the pectin by an ester, Poly (ethylene glycol) methyl ether functionalized LMP (LMP/PEGME-750)

Pectin Na salt made from LMP can be formed by reaction with 0.5M NaOH solution

TABLE 2
Example 5

| | | Total time to |
|---|---|---|
| | Solubility | reach the |
| Sample | (solute/solvent), wt % | saturation, h |
| LMP (DE = 8.9%) | (0.0714 g/9.9976 g), 0.714% | 24 hrs |
| LMP (DE = 9.9%) | (0.0614 g/9.9922 g), 0.614% | 24 hrs |
| Amidated LMP | (0.0566 g/9.9989 g), 0.566% | 24 hrs |
| (DE = 26%, Degree | | |
| of amidation = 22%) | | |
| HMP (DE = 71.5%), | (0.0219 g/9.9976 g), 0.219% | 72 hrs |
| Poly (ethylene glycol) | (0.0758 g/10.0064 g), 0.758% | 24 hrs |
| functionalized LMP | | |
| (LMP/PEG-200) | | |
| Poly(ethylene glycol) | (0.0769/9.9860), 0.770% | 24 hrs |
| methyl ether | | |
| functionalized LMP | | |
| (LMP/PEGME-750) | | |
| Pectin Na salt made from | (0.0645/9.9934), 0.645% | 24 hrs |
| LMP | | |

*No particles can be observed under the microscope after the solubility testing.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

Many variations and modifications may be made to the above-described embodiments. All such modifications and variations are intended to be included herein within the scope of this disclosure and protected by the following claims.

We claim:

1. A composition comprising:
a pectin compound having structure E 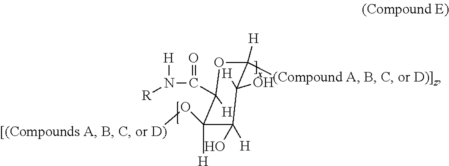
wherein R is a polyoxyalkyleneamine, wherein one or more of any one of compound structures A, B, C, or D can be included in compound E, wherein compound structure A, B, C, and D are selected from the following: 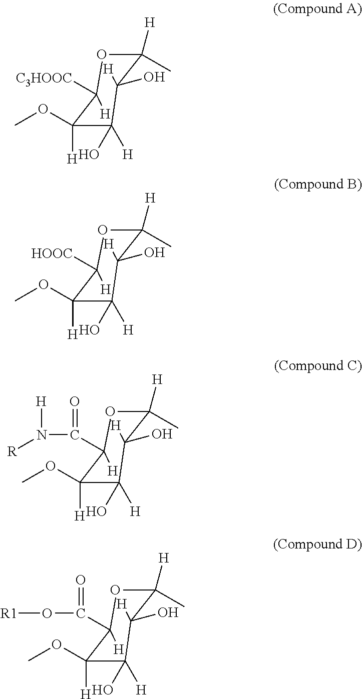
wherein each R1 is selected from an aliphatic group or a drug with an alcohol functionality, and wherein z is 300 to 800 and wherein x is 5 to 55.

2. The composition of claim 1, wherein R is structure F, 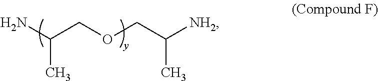
wherein y is 5 to 55.

3. The composition of claim 1, wherein the pectin compound is structure H,

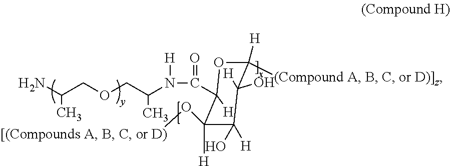

4. The composition of claim 1, further comprising an agent, wherein the agent is selected from: a drug, a pesticide, or a nutriceutical, with and amine or alcohol moiety; and a drug, a pesticide or a nutriceutical, that has similar solubility properties as the pectin compound.

5. The composition of claim 1, wherein ratio of the ester groups to the acid groups is about 10:90 to 85:15.

6. The composition of claim 1, wherein ratio of the amide groups to the acid groups is about 15:85 to 75:25.

7. The composition of claim 1, wherein the pectin compound has one or more of the following ratios: the ratio of the ester groups to the acid of about 25:75 to 75:25, the ratio of the amide groups to the acid groups of about 20:80 to 60:40, or the ratio of the ester groups of about 40:20 to 20:40.

8. A method of controlling the water solubility of a pectin compound, comprising:
adjusting the ratio of the ester groups to the acid groups on the pectin compound, wherein the ratio of the ester groups to the acid groups determines the water solubility of the pectin compound, wherein if the lower the ratio the higher the water solubility of the pectin compound and the higher the ratio the lower the water solubility of the pectin compound.

9. The method of claim 8, wherein adjusting includes reacting the pectin compound with a polyoxyalkyleneamine compound so that the polyoxyalkyleneamine compound displaces the ester group of the pectin compound.

10. The method of claim 8, wherein adjusting includes reacting the pectin compound with an aliphatic alcohol so that the carboxylic acid is converted into an ester group of the pectin compound.

11. The method of claim 9, wherein R is structure H, 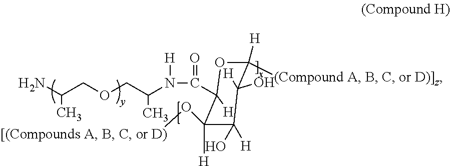
wherein y is 5 to 20.

12. The method of claim 9, wherein ratio of the ester groups to the acid groups is about 10:90 to 85:15.

13. A method of controlling the water solubility of a pectin compound, comprising:
adjusting the ratio of the amide groups to the acid groups on the pectin compound, wherein the ratio of the amide groups to the acid groups determines the water solubility of the pectin compound, wherein if the lower the ratio the higher the water solubility of the pectin compound and the higher the ratio the lower the water solubility of the pectin compound.

14. The method of claim 13, wherein ratio of the amide groups to the acid groups is about 15:85 to 75:25.

15. A method of controlling the water solubility of a pectin compound, comprising:
adjusting the ratio of one of: the amide groups to the acid groups, the ratio of the ester groups to the acid groups, or the ratio of the ester groups to the acid groups to the amide groups.

16. The method of claim 15, wherein adjusting includes reacting the pectin compound with a polyoxyalkyleneamine compound so that the polyoxyalkyleneamine compound displaces the ester group of the pectin compound.

17. The method of claim 15, wherein adjusting includes reacting the pectin compound with a polyoxyalkyleneamine compound so that the polyoxyalkyleneamine compound cleaves the ester group of the pectin compound forming an amide.

18. The method of claim 15, wherein adjusting includes reacting the pectin compound with a polyoxyalkyleneamine compound so that the polyoxyalkyleneamine compound is converted into the amide group of the pectin compound.

19. The method of claim 16, wherein R is structure F, 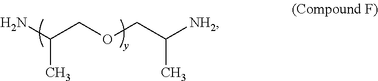
wherein y is 5 to 55.

20. The method of claim 15, wherein the ratio of the ester groups to the acid of about 25:75 to 75:25, wherein the ratio of the amide groups to the acid groups of about 20:80 to 60:40, or wherein the ratio of the ester groups to the amide groups of about 40:20 to 20:40.

21. A method of controlling the water solubility of an agent in a pectin compound, comprising:
altering the water solubility of the pectin compound to match the water solubility of the agent, wherein the water solubility is altered by adjusting the ratio of: the amide groups to the acid groups, the ratio of the ester groups to the acid groups, or the ratio of the ester groups to the acid groups to the amide groups.

22. The method of claim 21, wherein adjusting includes reacting the pectin compound with a polyoxyalkyleneamine compound so that the polyoxyalkyleneamine compound displaces the ester group of the pectin compound.

23. The method of claim 21, wherein adjusting includes reacting the pectin compound with a polyoxyalkyleneamine compound so that the polyoxyalkyleneamine compound cleaves the ester group of the pectin compound forming an amide.

24. The method of claim 21, wherein adjusting includes reacting the pectin compound with a polyoxyalkyleneamine compound so that the polyoxyalkyleneamine compound is converted into the amide group of the pectin compound.

25. The method of claim 24, wherein R is structure F, 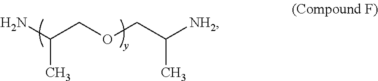
wherein y is 5 to 55.

26. The method of claim 21, wherein the ratio of the ester groups to the acid of about 25:75 to 75:25, wherein the ratio of the amide groups to the acid groups of about 20:80 to 60:40, or wherein the ratio of the ester groups of about 40:20 to 20:40.

27. A method of controlling the water solubility of a pectin compound, comprising:
adjusting the ratio of the ester groups to the acid groups on the pectin compound, wherein the ratio of the ester groups to the acid groups determines the water solubility of the pectin compound, wherein if the lower the ratio the lower the water solubility of the pectin compound and the higher the ratio the higher the water solubility of the pectin compound.

28. The method of claim 27, wherein the ratio of the ester groups to the acid of about 25:75 to 75:25.

29. A method of controlling the water solubility of a pectin compound, comprising:
adjusting the ratio of the amide groups to the acid groups on the pectin compound, wherein the ratio of the amide groups to the acid groups determines the water solubility of the pectin compound, wherein if the lower the ratio the lower the water solubility of the pectin compound and the higher the ratio the higher the water solubility of the pectin compound.

30. The method of claim 29, wherein the ratio of the amide groups to the acid groups of about 20:80 to 60:40
